SEQUENCE LISTING (104 sequences)

SEQ ID NO: 1 - DNA, 69 bp, Artificial
  Synthetic DNA
taatctagacatcatcattaattcctaatttttgttgacactctatcattgatagagttattttaccac

SEQ ID NO: 2 - DNA, 69 bp, Artificial
  Synthetic DNA
ttcttaatgtttttcatttcacttttctctatcactgatagggagtggtaaaataactctatcaatgat

SEQ ID NO: 3 - DNA, 29 bp, Artificial
  Synthetic DNA
tgaaaaacattaagaaaaaccaagttatg

SEQ ID NO: 4 - DNA, 21 bp, Artificial
  Synthetic DNA
aggacggtggccgttctaaag

SEQ ID NO: 5 - DNA, 35 bp, Artificial
  Synthetic DNA
aacggccaccgtcctcacatatacctgccgttcac

SEQ ID NO: 6 - DNA, 37 bp, Artificial
  Synthetic DNA
aataggggttccgcgtgtgcatgatctcctcgaaaag

SEQ ID NO: 7 - DNA, 27 bp, Artificial
  Synthetic DNA
cgcggaacccctatttgtttatttttc

SEQ ID NO: 8 - DNA, 38 bp, Artificial
  Synthetic DNA
atgatgtctagattagataaaagtaaagtgattaacag

SEQ ID NO: 9 - DNA, 20 bp, Artificial
  Synthetic DNA
caagcagatttatcgccagc

SEQ ID NO: 10 - DNA, 24 bp, Artificial
  Synthetic DNA
tggacggcagctgatcctgccagg

SEQ ID NO: 11 - DNA, 20 bp, Artificial
  Synthetic DNA
caagcagatttatcgccagc

SEQ ID NO: 12 - DNA, 19 bp, Artificial
  Synthetic DNA
gttgggtgacctgacgcag

SEQ ID NO: 13 - DNA, 44 bp, Artificial
  Synthetic DNA
ataatatactagtaacgtaaatactagttagtagatgatagttg

SEQ ID NO: 14 - DNA, 25 bp, Artificial
  Synthetic DNA
tgtatggatgggggtaatagaattg

SEQ ID NO: 15 - DNA, 42 bp, Artificial
  Synthetic DNA
acccccatccatacaagcatgtataacaaacactgatttttg

SEQ ID NO: 16 - DNA, 38 bp, Artificial
  Synthetic DNA
tcttaatgtttttcactgcaaaacttgtgcttgtacac

SEQ ID NO: 17 - DNA, 29 bp, Artificial
  Synthetic DNA
tgaaaaacattaagaaaaaccaagttatg

SEQ ID NO: 18 - DNA, 37 bp, Artificial
  Synthetic DNA
gcgtgacataactaatcatttcaagaaggtttcggag

SEQ ID NO: 19 - DNA, 26 bp, Artificial
  Synthetic DNA
ttagttatgtcacgcttacattcacg

SEQ ID NO: 20 - DNA, 59 bp, Artificial
  Synthetic DNA
aattgcccgactcatattaccctgttatccctaagcttgcaaattaaagccttcgagcg

SEQ ID NO: 21 - DNA, 18 bp, Artificial
  Synthetic DNA
atgagtcgggcaattccg

SEQ ID NO: 22 - DNA, 46 bp, Artificial
  Synthetic DNA
ctgggcctccatgtctatcgttaatatttcgtatgtgtattctttg

SEQ ID NO: 23 - DNA, 20 bp, Artificial
  Synthetic DNA
gacatggaggcccagaatac

SEQ ID NO: 24 - DNA, 24 bp, Artificial
  Synthetic DNA
ggttgtttatgttcggatgtgatg

SEQ ID NO: 25 - DNA, 41 bp, Artificial
  Synthetic DNA
cgaacataaacaaccatgggtaaggaaaagactcacgtttc

SEQ ID NO: 26 - DNA, 46 bp, Artificial
  Synthetic DNA
tattgtcagtactgattagaaaaactcatcgagcatcaaatgaaac

SEQ ID NO: 27 - DNA, 35 bp, Artificial
  Synthetic DNA
tcagtactgacaataaaaagattcttgttttcaag

SEQ ID NO: 28 - DNA, 27 bp, Artificial
  Synthetic DNA
cagtatagcgaccagcattcacatacg

SEQ ID NO: 29 - DNA, 48 bp, Artificial
  Synthetic DNA
atagcatacattatacgaagttatcccacacaccatagcttcaaaatg

SEQ ID NO: 30 - DNA, 37 bp, Artificial
  Synthetic DNA
caccgaaatcttcatcccttagattagattgctatgc

SEQ ID NO: 31 - DNA, 41 bp, Artificial
  Synthetic DNA
gctggtcgctatactgcgtgatttacatatactacaagtcg

SEQ ID NO: 32 - DNA, 48 bp, Artificial
  Synthetic DNA
aaaaacataagacaaattaccctgttatccctatgaccggatgaaacc

SEQ ID NO: 33 - DNA, 37 bp, Artificial
  Synthetic DNA
gggataacagggtaatggtacccaattcgccctatag

SEQ ID NO: 34 - DNA, 20 bp, Artificial
  Synthetic DNA
taccgcacagatgcgtaagg

SEQ ID NO: 35 - DNA, 44 bp, Artificial
  Synthetic DNA
ttacgcatctgtgcggtaaggaatcatagtttcatgattttctg

SEQ ID NO: 36 - DNA, 45 bp, Artificial
  Synthetic DNA
caggatgacgcctaaaaagattctctttttttatgatatttgtac

SEQ ID NO: 37 - DNA, 20 bp, Artificial
  Synthetic DNA
ttaggcgtcatcctgtgctc

SEQ ID NO: 38 - DNA, 35 bp, Artificial
  Synthetic DNA
cacactaaattaataatgaagatttcggtgatccc

SEQ ID NO: 39 - DNA, 33 bp, Artificial
  Synthetic DNA
tattaatttagtgtgtgtatttgtgtttgtgtg

SEQ ID NO: 40 - DNA, 40 bp, Artificial
  Synthetic DNA
gcagattgtactgagagtacgacatcgtcgaatatgattc

SEQ ID NO: 41 - DNA, 26 bp, Artificial
  Synthetic DNA
actctcagtacaatctgctctgatgc

SEQ ID NO: 42 - DNA, 38 bp, Artificial
  Synthetic DNA
ttactagtatattatgctccagcttttgttccctttag

SEQ ID NO: 43 - DNA, 48 bp, Artificial
  Synthetic DNA
tcaaggagaaaaaaccagcatgtataacaaacactgatttttgttttg

SEQ ID NO: 44 - DNA, 38 bp, Artificial
  Synthetic DNA
cggcttctaatccgtgctccagcttttgttccctttag

SEQ ID NO: 45 - DNA, 20 bp, Artificial
  Synthetic DNA
acggattagaagccgccgag

SEQ ID NO: 46 - DNA, 29 bp, Artificial
  Synthetic DNA
ggttttttctccttgacgttaaagtatag

SEQ ID NO: 47 - DNA, 52 bp, Artificial
  Synthetic DNA
catgttctttcctgcgttattaagacccactttcacatttaagttgtttttc

SEQ ID NO: 48 - DNA, 45 bp, Artificial
  Synthetic DNA
ctatcactgatagggagattttcacttttctctatcactgatagg

SEQ ID NO: 49 - DNA, 69 bp, Artificial
  Synthetic DNA
tccctatcagtgatagagattgacatccctatcagtgatagagatactgagcacatcagcaggacgcac

SEQ ID NO: 50 - DNA, 69 bp, Artificial
  Synthetic DNA
aagttaaacaaaattatttctagctttctcctctttaatgaattcggtcagtgcgtcctgctgatgtgc

SEQ ID NO: 51 - DNA, 69 bp, Artificial
  Synthetic DNA
gaaataattttgtttaactttaagaaggagatatacataatgaaaaacattaagaaaaaccaagttatg

SEQ ID NO: 52 - DNA, 100 bp, Artificial
  Synthetic DNA
taggccagtacctcccgcttatgtatttactcgtaggatttgcttcgttcgatcagcacaagccttcaaagatgatcatttcaagaaggtttcggaggag

SEQ ID NO: 53 - DNA, 98 bp, Artificial
  Synthetic DNA
gggaggtactggcctagcgtcgtggcccgggagagacagtttagtagtgactcgcggccagttagggataacagggtaatatggcgattgcaattggc

SEQ ID NO: 54 - DNA, 61 bp, Artificial
  Synthetic DNA
caatcacagggcgggaaataagctacaattaacgccaaaaaattatagagtcgcaacggcc

SEQ ID NO: 55 - DNA, 59 bp, Artificial
  Synthetic DNA
tcccgccctgtgattgaggcgggatggtgtccccacagtatgaccgcactaacagaagg

SEQ ID NO: 56 - DNA, 67 bp, Artificial
  Synthetic DNA
catacatttctccacgggacccacagtcgtagatgcgtaaaatcaaccttggtaagtatccaaatcc

SEQ ID NO: 57 - DNA, 60 bp, Artificial
  Synthetic DNA
cgtggagaaatgtatgaaaccctgtatggagagtgattcagtccagccaggacagaaatg

SEQ ID NO: 58 - DNA, 105 bp, Artificial
  Synthetic DNA
ttctcccaagtgtacgatatcacacctagcgccgtgcaaaaaaaaccacgtcaaataatcaaggcgccttgaatgctcgagggttatttgccgactaccttggtg

SEQ ID NO: 59 - DNA, 73 bp, Artificial
  Synthetic DNA
cgtacacttgggagaagtcagatacgattgcggctcagtatgacgatttttgataattatgaagtgtggtttg

SEQ ID NO: 60 - DNA, 75 bp, Artificial
  Synthetic DNA
cggcagtaccggatcctaaagccgattcaagaaaaattaccctgttatccctattagcgacgaaacccgtaatac

SEQ ID NO: 61 - DNA, 61 bp, Artificial
  Synthetic DNA
ggatccggtactgccgacgcactttagaacggccaccgtcctggtccttttcatcacgtgc

SEQ ID NO: 62 - DNA, 24 bp, Artificial
  Synthetic DNA
taacgcaggaaagaacatgtgagc

SEQ ID NO: 63 - DNA, 45 bp, Artificial
  Synthetic DNA
ggcaagcgatccgtcctaggcaagagagaagacccagagatgttg

SEQ ID NO: 64 - DNA, 56 bp, Artificial
  Synthetic DNA
aggataactggccatttttagtttatgtatgtgttttttgtagttatagatttaag

SEQ ID NO: 65 - DNA, 22 bp, Artificial
  Synthetic DNA
atggccagttatccttgtcacc

SEQ ID NO: 66 - DNA, 23 bp, Artificial
  Synthetic DNA
tcaattagcttcccccatttctc

SEQ ID NO: 67 - DNA, 38 bp, Artificial
  Synthetic DNA
ggggaagctaattgagagccagtttattcttgccatcc

SEQ ID NO: 68 - DNA, 43 bp, Artificial
  Synthetic DNA
tgaaactatgattcctcgatcaatgcgaaattccaactatttc

SEQ ID NO: 69 - DNA, 30 bp, Artificial
  Synthetic DNA
aggaatcatagtttcatgattttctgttac

SEQ ID NO: 70 - DNA, 22 bp, Artificial
  Synthetic DNA
ggacggatcgcttgcctgtaac

SEQ ID NO: 71 - DNA, 20 bp, Artificial
  Synthetic DNA
acggattagaagccgccgag

SEQ ID NO: 72 - DNA, 29 bp, Artificial
  Synthetic DNA
ggttttttctccttgacgttaaagtatag

SEQ ID NO: 73 - DNA, 48 bp, Artificial; identical to SEQ ID NO: 43
  Synthetic DNA

SEQ ID NO: 74 - DNA, 38 bp, Artificial; identical to SEQ ID NO: 16
  Synthetic DNA

SEQ ID NO: 75 - DNA, 29 bp, Artificial
  Synthetic DNA
tgaaaaacattaagaaaaaccaagttatg

SEQ ID NO: 76 - DNA, 37 bp, Artificial; identical to SEQ ID NO: 18
  Synthetic DNA

SEQ ID NO: 77 - DNA, 26 bp, Artificial
  Synthetic DNA
ttagttatgtcacgcttacattcacg

SEQ ID NO: 78 - DNA, 59 bp, Artificial; identical to SEQ ID NO: 20
  Synthetic DNA

SEQ ID NO: 79 - DNA, 18 bp, Artificial
  Synthetic DNA
atgagtcgggcaattccg

SEQ ID NO: 80 - DNA, 46 bp, Artificial; identical to SEQ ID NO: 22
  Synthetic DNA

SEQ ID NO: 81 - DNA, 20 bp, Artificial
  Synthetic DNA
gacatggaggcccagaatac

SEQ ID NO: 82 - DNA, 24 bp, Artificial
  Synthetic DNA
ggttgtttatgttcggatgtgatg

SEQ ID NO: 83 - DNA, 41 bp, Artificial; identical to SEQ ID NO: 25
  Synthetic DNA

SEQ ID NO: 84 - DNA, 46 bp, Artificial; identical to SEQ ID NO: 26
  Synthetic DNA

SEQ ID NO: 85 - DNA, 35 bp, Artificial; identical to SEQ ID NO: 27
  Synthetic DNA

SEQ ID NO: 86 - DNA, 27 bp, Artificial
  Synthetic DNA
cagtatagcgaccagcattcacatacg

SEQ ID NO: 87 - DNA, 48 bp, Artificial; identical to SEQ ID NO: 29
  Synthetic DNA

SEQ ID NO: 88 - DNA, 37 bp, Artificial; identical to SEQ ID NO: 30
  Synthetic DNA

SEQ ID NO: 89 - DNA, 41 bp, Artificial; identical to SEQ ID NO: 31
  Synthetic DNA

SEQ ID NO: 90 - DNA, 48 bp, Artificial; identical to SEQ ID NO: 32
  Synthetic DNA

SEQ ID NO: 91 - DNA, 37 bp, Artificial; identical to SEQ ID NO: 33
  Synthetic DNA

SEQ ID NO: 92 - DNA, 20 bp, Artificial
  Synthetic DNA
taccgcacagatgcgtaagg

SEQ ID NO: 93 - DNA, 44 bp, Artificial; identical to SEQ ID NO: 35
  Synthetic DNA

SEQ ID NO: 94 - DNA, 45 bp, Artificial; identical to SEQ ID NO: 36
  Synthetic DNA

SEQ ID NO: 95 - DNA, 20 bp, Artificial
  Synthetic DNA
ttaggcgtcatcctgtgctc

SEQ ID NO: 96 - DNA, 35 bp, Artificial; identical to SEQ ID NO: 38
  Synthetic DNA

SEQ ID NO: 97 - DNA, 33 bp, Artificial; identical to SEQ ID NO: 39
  Synthetic DNA

SEQ ID NO: 98 - DNA, 40 bp, Artificial; identical to SEQ ID NO: 40
  Synthetic DNA

SEQ ID NO: 99 - DNA, 26 bp, Artificial
  Synthetic DNA
actctcagtacaatctgctctgatgc

SEQ ID NO: 100 - DNA, 38 bp, Artificial; identical to SEQ ID NO: 44
  Synthetic DNA

SEQ ID NO: 101 - DNA, 48 bp, Artificial; identical to SEQ ID NO: 43
  Synthetic DNA

SEQ ID NO: 102 - DNA, 38 bp, Artificial; identical to SEQ ID NO: 44
  Synthetic DNA

SEQ ID NO: 103 - DNA, 21 bp, Artificial
  Synthetic DNA
ggtccttttcatcacgtgcta

SEQ ID NO: 104 - DNA, 29 bp, Artificial
  Synthetic DNA
ggttttttctccttgacgttaaagtatag

(19) United States
(12) Patent Application Publication
(10) Pub. No.: US 2024/0043875 A1
(43) Pub. Date: Feb. 8, 2024

(54) A PLASMID FOR TRANSFORMATION, A METHOD FOR PRODUCING A TRANSFORMANT USING THE SAME AND A METHOD OF TRANSFORMATION

(71) Applicant: TOYOTA JIDOSHA KABUSHIKI KAISHA, Toyota-shi, Aichi (JP)

(72) Inventor: Toru ONISHI, Toyota-shi, Aichi (JP)

(21) Appl. No.: 18/258,909

(22) PCT Filed: Dec. 21, 2021
(86) PCT No.: PCT/JP2021/047338

§ 371 Date: Jun. 22, 2023

(30) Foreign Application Priority Data
Dec. 23, 2020 (JP) 2020-213177

(51) Int. Cl. C12N 15/90; C12N 15/11; C12N 15/70; C12N 15/81; C12N 9/22

(52) U.S. Cl. CPC C12N 15/902; C12N 15/11; C12N 15/70; C12N 15/81; C12N 9/22; C12N 2310/20; C12N 2800/80; C12N 2800/101; C12N 2800/102

(57) ABSTRACT

This invention is intended to produce a stable transformant comprising a gene of interest incorporated into the genome in a simple and efficient manner. Such transformant comprises a site into which a gene of interest is to be incorporated, a pair of homologous recombination sequences, a pair of endonuclease target sequences, and a counter selection marker.

### TECHNICAL FIELD

The present invention relates to a plasmid for transformation that is used upon introduction of a gene of interest into a host, a method of producing a transformant using the plasmid for transformation, and a transformation method using the plasmid for transformation.

### BACKGROUND ART

In general, a technique of introducing a gene of interest into a host cell from the outside is referred to as transformation or gene recombination, and a cell into which the gene of interest is introduced is referred to as a transformant or a recombinant. By efficiently producing such a transformant utilizing a transformation technique, acceleration and/or efficiency of microbial metabolic engineering can be promoted, for example, utilizing a synthetic biological technique. Herein, the synthetic biological technique means a technique of promptly turning a cycle consisting of the designing, construction, evaluation, and learning of a production host. In synthetic biology involving the use of a yeast or prokaryotic host, in particular, it is important to efficiently construct a host, namely, to efficiently produce a recombinant yeast.

Transformation using a yeast as a host is roughly classified into a method involving the use of a circular plasmid into which a gene of interest is incorporated and a method involving the use of a linear vector comprising a gene of interest. It is easy to introduce a gene of interest into a yeast using a circular plasmid, and a transformed yeast can be produced at a high efficiency of approximately 10⁻² (Non-Patent Literature 1). On the other hand, when a gene of interest is introduced into a yeast using a linear vector, it is necessary to incorporate the gene of interest into the genome via homologous recombination. Thus, a transformed yeast can be produced only at an efficiency of approximately 10⁻⁶ (Non-Patent Literature 2).

As described above, the method of introducing a gene of interest into a yeast using a circular plasmid is highly efficient. However, such a circular plasmid may be detached in some case, and thus, a stable recombinant yeast cannot be produced. On the other hand, in the method of introducing a gene of interest into a yeast using a linear vector, the gene of interest is stably incorporated into the genome. However, as described above, this method is not considered to be highly efficient.

In order to improve the efficiency of introducing a gene of interest into the genome, known is a technique, in which the target sequence of target-specific endonuclease such as homing endonuclease has previously been introduced into a predetermined introduction site in the genome, and then, the double strands at the site have previously been cleaved (Non-Patent Literature 2). Moreover, also known is a technique, in which the double strands of a predetermined introduction site in the genome have previously been cleaved by applying a technique of cleaving any given nucleotide sequence, such as CRISPR-Cas9 or TALEN, instead of the target-specific endonuclease (Non-Patent Literature 3). Hence, it is possible to improve homologous recombination efficiency to approximately 10⁻² to 10⁻¹ by previously cleaving the double strands at the site into which a gene of interest is to be introduced.

However, in these methods of improving the efficiency of introducing a gene of interest, it has been necessary to previously introduce an endonuclease target sequence into a predetermined introduction site in the genome, or it has been necessary to produce guide RNA or the like corresponding to the target site. Thus, these methods of improving the efficiency of introducing a gene of interest are complicated, and require various steps, in addition to production of a DNA fragment for homologous recombination containing a gene of interest and the subsequent transformation using the produced DNA fragment.

In addition, Patent Literature 1 discloses a plasmid comprising a selection marker having an intron configured to sandwich a homing endonuclease recognition sequence with telomere seed sequences. In the case of the plasmid disclosed in Patent Literature 1, as a result of the expression of the homing endonuclease, the circular plasmid can be converted to linear molecules and can be stably present because of the telomere seed sequence at the terminus.

In a prokaryotic cell such as Escherichia coli, in addition, efficiency of transformation involving the use of a plasmid is very high. However, efficiency of genome modification via homologous recombination involving the use of a circular or linear vector is very poor, compared with yeasts. In order to improve efficiency of homologous recombination, a method in which an E. coli strain comprising a plasmid containing a Red recombinase operon, which involves in a lambda phage homologous recombination mechanism, introduced in advance is prepared, and a linear vector is introduced into such E. coli strain is a standard technique (Non-Patent Literature 4). Such technique is employed for Lactobacillusor Coryncbacterium (Non-Patent Literature 5 and Non-Patent Literature 6). According to such technique, however, it is necessary to conduct transformation two times, and, disadvantageously, this complicates the procedure.

### CITATION LIST

### Patent Literature

PTL 1: US 2016/0017344

### Non Patent Literature

NPL 1: Gietz, R. D., et al., “High-efficiency yeast transformation using the LiAc/SS carrier DNA/PEG method,” Nature Protocols, 2, 2007: 31-34
NPL 2: Storici, F., et al., “Chromosomal site-specific double-strand breaks are efficiently targeted for repair by oligonucleotides in yeast,” Proc. Natl. Acad. Sci., U.S.A., 100, 2003: 14994-14999
NPL 3: DiCarlo, J. E., et al., “Genome engineering in Saccharomyces cerevisiae using CRISPR-Cas systems,” Nucleic Acids Res., 41, 2013: 4336-4343
NPL 4: Zhang, Y., et al., “A new logic for DNA engineering using recombination in Escherichia coli,” Nature Genetics 20, 1998: 123-128
NPL 5: Peng, Y., et al., “Prophage recombinases-mediated genome engineering in Lactobacillus plantarum,” Microb. Cell Fact., 14, 2015: 154{NPL 6}Huang, Y., et al., “Recombineering using RecET in Corynebacterium glutamicum ATCC14067 via a selfexcisable cassette,” Sci. Rep., 7, 2017: 7916

### SUMMARY OF INVENTION

### Technical Problem

However, all of the aforementioned methods have been problematic in that a stable transformant, in which a gene of interest is incorporated into the genome, cannot be simply and efficiently produced according to the methods. Under the aforementioned circumstances, it is an object of the present invention to provide a plasmid for transformation capable of simply and efficiently producing a stable transformant, in which a gene of interest is incorporated into the genome, a method of producing a transformant using the same, and a transformation method.

### Solution to Problem

The present invention that dissolves the aforementioned problem includes the following.
(1) A plasmid for transformation comprising a site into which a gene of interest is to be incorporated, a pair of homologous recombination sequences sandwiching the site, a pair of endonuclease target sequences sandwiching the pair of homologous recombination sequences, and a counter selection marker.
(2) The plasmid for transformation according to (1), which further comprises a target-specific endonuclease gene specifically cleaving the double strands of the endonuclease target sequences in an expressible manner.
(3) The plasmid for transformation according to (2), wherein the target-specific endonuclease gene is a homing endonuclease gene.
(4) The plasmid for transformation according to (3), wherein the endonuclease target sequence is specifically recognized by homing endonuclease.
(5) The plasmid for transformation according to (2), which further comprises an inducible promoter regulating the expression of the target-specific endonuclease gene.
(6) The plasmid for transformation according to any one of the above (1) to (5), which comprises the gene of interest that is incorporated into the site.
(7) A method of producing a transformant, comprising steps of:
introducing the plasmid for transformation according to (6) into a host; and selecting a transformant, in which the gene of interest comprised in the plasmid for transformation is incorporated into the genome of the host via the homologous recombination sequences comprised in the plasmid for transformation, and in which the gene of interest is then expressed therein,
wherein the counter selection marker functions to induce the death of a host comprising the plasmid for transformation comprising the gene of interest incorporated therein.
(8) A transformation method comprising a step of introducing the plasmid for transformation according to (6) into a host, wherein the gene of interest comprised in the plasmid for transformation is expressed in the host and the counter selection marker functions to induce the death of a host comprising the plasmid for transformation comprising the gene of interest incorporated therein.
(9) The transformation method according to (8), wherein the gene of interest is incorporated into the genome of the host via the homologous recombination sequences comprised in the plasmid for transformation.

### Advantageous Effects of Invention

With the use of the plasmid for transformation according to the present invention, the counter selection marker functions to induce the death of a host in which a region comprising a gene of interest has not been cleaved from the plasmid for transformation. Thus, a transformant in which a gene of interest is incorporated into a host genome can be efficiently produced.

Moreover, the method of producing a transformant according to the present invention utilizes the plasmid for transformation according to the present invention. Thus, the counter selection marker functions to induce the death of a host in which a region comprising a gene of interest has not been cleaved from the plasmid for transformation, and a transformant in which a gene of interest is incorporated into the host genome can be efficiently produced.

Furthermore, the transformation method of the present invention utilizes the plasmid for transformation according to the present invention. Thus, the counter selection marker functions to induce death of a host in which a region comprising a gene of interest has not been cleaved from the plasmid for transformation, and excellent transformation efficiency can be achieved upon the production of a transformant in which a gene of interest is incorporated into the host genome.

### BRIEF DESCRIPTION OF DRAWINGS

FIG. 1 is a configuration diagram schematically showing main parts of the plasmid for transformation according to the present invention.

FIG. 2 is a configuration diagram schematically showing one configuration example of the plasmid for transformation according to the present invention.

FIG. 3 is a configuration diagram schematically showing a mechanism of incorporating a gene of interest into a genome using the plasmid for transformation according to the present invention.

FIG. 4 is a configuration diagram schematically showing the plasmid for transformation prepared in Example 2.

### DESCRIPTION OF EMBODIMENTS

Hereinafter, the present invention will be described in more detail using drawings and examples.

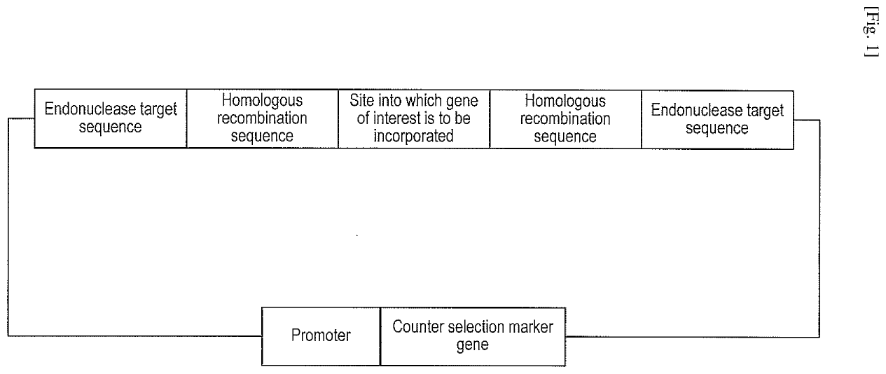

As shown in FIG. 1, the plasmid for transformation according to the present invention comprises a site into which a gene of interest is to be incorporated, a pair of homologous recombination sequences sandwiching (interposing) the site, a pair of endonuclease target sequences sandwiching (interposing) the pair of homologous recombination sequences, and a counter selection marker. In other words, having the sense strand of the gene of interest as a reference, the plasmid for transformation comprises, from the 5′-side to the 3′-side, one endonuclease target sequence (which may also be referred to as a “first endonuclease target sequence”), one homologous recombination sequence (which may also be referred to as a “first homologous recombination sequence”), a site into which a gene of interest is to be incorporated, the other homologous recombination sequence (which may also be referred to as a “second homologous recombination sequence”), and the other endonuclease target sequence (which may also be referred to as a “second endonuclease target sequence”) in this order. The counter selection marker may be comprised in the plasmid for transformation independently of the site into which a gene of interest is to be incorporated, the pair of homologous recombination sequences sandwiching the site, and the pair of endonuclease target sequences sandwiching the pair of homologous recombination sequences.

The term “counter selection marker” used herein refers to, for example, a gene that is expressed in a cell to induce death of the cell, and such gene is used as a marker. In this case, a cell comprising a counter selection marker is induced to die upon expression of a counter selection marker gene under particular conditions. In the presence of both a cell with a counter selection marker and a cell without a counter selection marker, accordingly, a cell that has grown under particular conditions can be selected as a cell without a selection marker.

A counter selection marker may be, for example, a gene that has functions of inducing cell death when gene expression is suppressed under particular conditions. When a counter selection marker functions, expression of a counter selection marker gene is suppressed under particular conditions, and the cell is induced to die. In the presence of both a cell with a counter selection marker and a cell without a counter selection marker, accordingly, a cell that has grown under particular conditions can be selected as a cell without a selection marker.

When an E. coli host is used, specifically, the sacB gene derived from Bacillus subtiliscan be used as a counter selection marker. A sacB gene product, levansucrase, has activity of converting sucrose to levan. Gram-negative bacteria, such as Escherichia coli, are induced to die upon accumulation of levan in the periplasm layer. Thus, the sacB gene can be used as a counter selection marker.

Another example of a counter selection marker is a variant of an alpha-subunit of phenylalanyl tRNA synthetase (PheS). A PheS variant incorporates a phenylalanine analog, which is 4-chloro-D,L-phenylalanine. Thus, a cell in which a PheS variant is expressed is not capable of synthesizing a normal polypeptide. Because a normal polypeptide cannot be synthesized, a cell in which the PheS variant is expressed is induced to die in the presence of 4-chloro-D,L-phenylalanine. By introducing an amino acid analog into a biosynthesized protein molecule, as described above, functions thereof would be damaged, and the cell can be induced to die. A variant gene that can be used in such method can be used as a counter selection marker.

Another example of a counter selection marker is a thymidine kinase gene. The thymidine kinase gene converts 5-fluoro-2-deoxyuridine (5FU) into a toxic metabolite, 5-fluorodeoxyuridine-5′-monophosphate, and inhibits thymine biosynthesis by inhibiting a thymidylate synthase. Accordingly, a cell in which the thymidine kinase gene is expressed is induced to die upon inhibition of thymine biosynthesis in the presence of 5FU.

In addition, a temperature-sensitive variant gene of a replication origin of the pSC101 plasmid (RepA) can be used as a counter selection marker. By subjecting a cell that carries a plasmid comprising such temperature-sensitive variant gene to culture at temperature over 37 degrees C., growth of the cell is inhibited. By transforming a cell with the use of a plasmid comprising such temperature-sensitive variant gene and performing culture in the temperate range described above, a cell from which a plasmid is detached can be selectively grown.

Further, a toxin-antitoxin system can also be used as a counter selection marker. When an antitoxin gene is expressed, in general, cell death induced by expression of a toxin gene is inhibited. By inhibiting antitoxin gene expression, accordingly, effects achieved by toxin gene expression can be made apparent, and cell death can be induced. By designing an antisense RNA that targets the endogenous antitoxin gene as a nucleotide sequence complementary to a part of mRNA of the antitoxin gene and inducing antisense RNA in a condition-specific manner, for example, antitoxin gene translation can be inhibited. As a result, the cell death can be induced upon toxin gene expression.

In the plasmid for transformation, “a site into which a gene of interest is to be incorporated” is a region into which a nucleic acid fragment comprising a gene of interest is to be incorporated. Accordingly, such a site into which a gene of interest is to be incorporated is not limited to a specific nucleotide sequence, and can be, for example, one or multiple restriction enzyme target sequences. Moreover, the term “a gene of interest” means a nucleic acid to be introduced into a host genome. Accordingly, such a gene of interest is not limited to a nucleotide sequence encoding a specific protein, and includes nucleic acids consisting of all types of nucleotide sequences, such as a nucleotide sequence encoding siRNA, etc., a nucleotide sequence of a transcriptional regulatory region that regulates the transcription period of a transcriptional product and the production amount thereof, such as a promoter or an enhancer, and a nucleotide sequence encoding transfer RNA (tRNA), ribosome RNA (rRNA), etc.

Furthermore, such a gene of interest is preferably incorporated into the above-described site in an expressible manner. In an expressible manner, a gene of interest is linked to a predetermined promoter and is then incorporated into the above-described site, so that the gene of interest can be expressed under the control of the promoter in a host organism.

In addition, a promoter and a terminator, and as desired, a cis element such as an enhancer, a splicing signal, a poly A addition signal, a selection marker, a ribosomal binding sequence (SD sequence), and the like can be linked to such a gene of interest. Examples of the selection marker include antibiotic resistance genes such as an ampicillin resistance gene, a kanamycin resistance gene, and a hygromycin resistance gene.

The term “a pair of homologous recombination sequences” means a pair of nucleic acid regions having homology to a certain region in a host genome. Such a pair of homologous recombination sequences each cross with the host genome having homology to the homologous recombination sequences, so that a gene of interest sandwiched with the pair of homologous recombination sequences can be incorporated into the host genome. Accordingly, such a pair of homologous recombination sequences are not particularly limited to specific nucleotide sequences, and can be, for example, nucleotide sequences having high homology to the upstream region and the downstream region of a certain gene present in the host genome. In this case, if homologous recombination takes place between the plasmid for transformation and the host genome, the gene is deleted from the host genome. As such, the success or failure of homologous recombination can be determined by observing a phenotype caused by the deletion of the gene.

For example, such a pair of homologous recombination sequences can be a region upstream of the coding region of an ADE1 gene associated with an adenine biosynthesis pathway, and a region downstream of the coding region of the ADE1 gene. In this case, if homologous recombination takes place between the pair of homologous recombination sequences and the host genome, an intermediate metabolite of adenine, 5-aminoimidazole riboside, is accumulated, and a transformant is colored red due to the polymerized polyribosylaminoimidazole. Accordingly, by detecting this red color, it can be determined that homologous recombination has taken place between the pair of homologous recombination sequences and the host genome.

Herein, the pair of homologous recombination sequences has high sequence identity to the recombination region in the host genome, to such an extent that they can be homologously recombined (can cross) with each other. The identity between the nucleotide sequences of individual regions can be calculated using conventional sequence comparison software “blastn,” etc. The nucleotide sequences of individual regions may have an identity of 60% or more, and the sequence identity is preferably 80% or more, more preferably 90% or more, particularly preferably 95% or more, and the most preferably 99% or more.

Still further, such a pair of homologous recombination sequences may have the same length, or may each have different lengths. The lengths of such a pair of homologous recombination sequences are not particularly limited, as long as the lengths are sufficient for possible homologous recombination (possible crossing). The length of each of the pair of homologous recombination sequences is, for example, preferably 0.1 kb to 3 kb, more preferably 0.5 kb to 3 kb, and particularly preferably 0.5 kb to 2 kb.

By the way, the plasmid for transformation according to the present invention comprises endonuclease target sequences outside of the aforementioned pair of homologous recombination sequences (i.e., outside of the aforementioned pair of homologous recombination sequences, when the gene of interest sandwiched by the pair of homologous recombination sequences is defined to be inside). The term “endonuclease target sequence” means a nucleotide sequence recognized by endonuclease.

The endonuclease is not particularly limited, and it extensively means an enzyme having activity of recognizing a predetermined nucleotide sequence and cleaving double-stranded DNA. Examples of the endonuclease include restriction enzymes, homing endonuclease, Cas9 nuclease, meganuclease (MN), zinc finger nuclease (ZFN), and transcriptional activation-like effector nuclease (TALEN). Moreover, the term “homing endonuclease” includes both endonuclease encoded by an intron (with the prefix “I-”) and endonuclease included in an intein (with the prefix “PI-”). More specific examples of the homing endonuclease include I-Ceu I, I-Sce I, I-Onu I, PI-Psp I, and PI-Sce I. Besides, target sequences specifically recognized by these specific endonucleases, namely, endonuclease target sequences, are known, and a person skilled in the art could appropriately acquire such endonuclease target sequences.

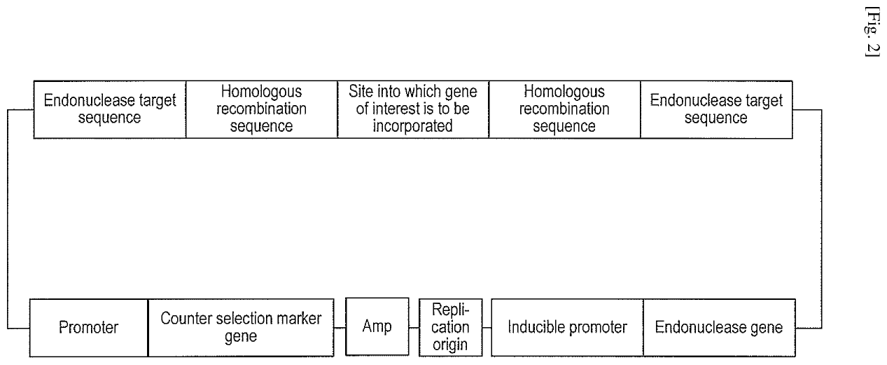

Moreover, as shown in FIG. 2, the plasmid for transformation according to the present invention may comprise an inducible promoter and an endonuclease gene. For the expression of an endonuclease gene, not only an inducible promoter, but also a constitutive expression promoter may be used.

This endonuclease gene encodes an enzyme having activity of specifically recognizing the aforementioned pair of endonuclease target sequences and cleaving the double strands. That is, examples of the endonuclease gene include a restriction enzyme gene, a homing endonuclease gene, a Cas9 nuclease gene, a meganuclease gene, a zinc finger nuclease gene, and a transcriptional activation-like effector nuclease gene.

The inducible promoter means a promoter having functions of inducing expression under specific conditions. Examples of the inducible promoter include, but are not particularly limited to, a promoter inducing expression in the presence of a specific substance, a promoter inducing expression under specific temperature conditions, and a promoter inducing expression in response to various types of stresses. The used promoter can adequately be selected depending on a host to be transformed.

Examples of the inducible promoter include galactose inducible promoters such as GAL1 and GAL10, Tet-on/Tet-off system promoters inducing expression with the addition or removal of tetracycline or a derivative thereof, and promoters of genes encoding heat shock proteins (HSP) such as HSP10, HSP60, and HSP90. In addition, as such an inducible promoter, a CUP1 promoter that activates with the addition of copper ions can also be used. Furthermore, when the host is a prokaryotic cell such as Escherichia coli, examples of the inducible promoter include a lac promoter inducing expression with IPTG, a cspA promoter inducing expression by cold shock, and an araBAD promoter inducing expression with arabinose.

Further, the method of controlling the expression of an endonuclease gene is not limited to a method involving the use of a promoter such as an inducible promoter or a consititutive expression promoter. For example, a method involving the use of DNA recombinase may be applied. An example of the method of turning the expression of a gene ON and OFF with the use of DNA recombinase may be a FLEx switch method (A FLEX Switch Targets Channelrhodopsin-2 to Multiple Cell Types for Imaging and Long-Range Circuit Mapping. Atasoy et al., The Journal of Neuroscience, 28, 7025-7030, 2008). According to the FLEx switch method, recombination to change the direction of a promoter sequence is caused by DNA recombinase, so that the expression of a gene can be turned ON and OFF.

On the other hand, the plasmid for transformation according to the present invention can be produced based on a conventional, available plasmid. Examples of such a plasmid include: YCp-type E. coli-yeast shuttle vectors, such as pRS413, pRS414, pRS415, pRS416, YCp50, pAUR112, and pAUR123; YEp-type E. coli-yeast shuttle vectors, such as pYES2 and YEp13; YIp-type E. coli-yeast shuttle vectors, such as pRS403, pRS404, pRS405, pRS406, pAUR101, and pAUR135; Escherichia coliderived plasmids (e.g., ColE-type plasmids, such as pBR322, pBR325, pUC18, pUC19, pUC118, pUC119, pTV118N, pTV119N, pBluescript, pHSG298, pHSG396, and pTrc99A; p15A-type plasmids, such as pACYC177 and pACYC184; and pSC101-type plasmids, such as pMW118, pMW119, pMW218, and pMW219); Agrobacterium-derived plasmids (e.g., pBI101); and Bacillus subtilis-derived plasmids (e.g., pUB110 and pTP5).

Moreover, the plasmid for transformation according to the present invention may further comprise a replication origin, an autonomously replicating sequence (ARS), and a centromere sequence (CEN). The plasmid for transformation comprises these elements, so that it can stably replicate after it has been introduced into a host cell. In addition, the plasmid for transformation according to the present invention may comprise a selection marker. The selection marker is not particularly limited, and examples of the selection marker include a drug resistance marker gene and an auxotrophic marker gene. The plasmid for transformation comprises these selection markers, so that a host cell into which the plasmid for transformation has been introduced can be efficiently selected.

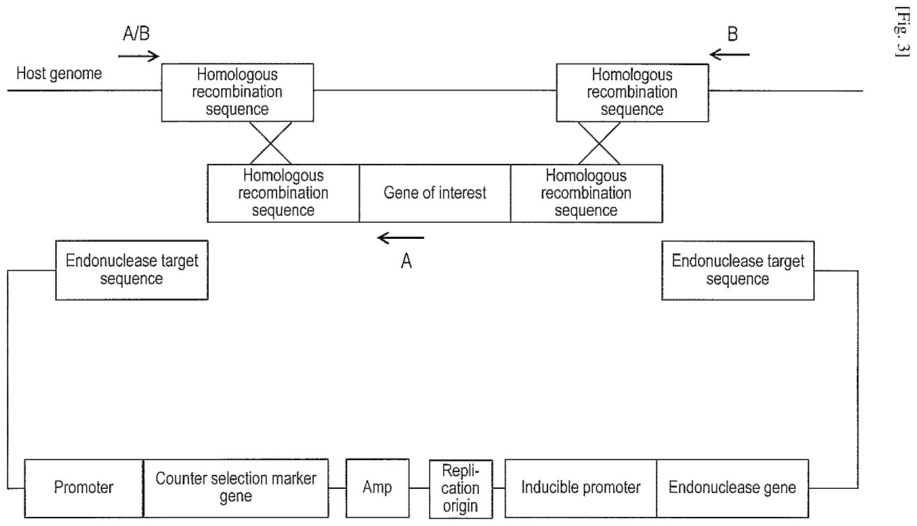

By using the thus configured plasmid for transformation, a stable transformant in which a gene of interest is incorporated into the genome can be simply and efficiently produced. To produce a transformant, at the outset, a gene of interest is incorporated into a site into which such a gene of interest is to be incorporated (FIG. 1). A plasmid for transformation comprising the gene of interest is then introduced into a host cell according to a common method. Thereafter, as schematically shown in FIG. 3, the double stands of a pair of endonuclease target sequences are cleaved by endonuclease that has been expressed under the control of an inducible promoter, so that a nucleic acid fragment comprising the gene of interest sandwiched with the pair of homologous recombination sequences is cleaved out. A pair of homologous recombination sequences in the thus cleaved nucleic acid fragment cross with homologous recombination sequences in the host genome, and the gene of interest is then incorporated into the genome. Thereby, a stable transformant in which the gene of interest is incorporated into the genome can be produced.

Herein, the method of introducing the plasmid for transformation into which the gene of interest has been incorporated into a host cell is not particularly limited, and conventional methods, such as a calcium chloride method, a competent cell method, a protoplast or spheroplast method, or an electrical pulse method, can be adequately employed. When the plasmid for transformation has a selection marker, the host cell into which the plasmid for transformation has been introduced can then be selected using the selection marker.

In order to allow endonuclease to express under the control of an inducible promoter, in addition, conditions are adequately determined depending on the type of the inducible promoter. When a galactose inducible promoter such as GAL1 or GAL10 is used as such an inducible promoter, for example, galactose is added to a medium for use in the culture of the host cell into which the plasmid for transformation has been introduced, or the host cell is transferred to a galactose-containing medium and is then cultured, so that the expression of the endonuclease can be induced. When a promoter of a gene encoding a heat shock protein (HSP) is used as such an inducible promoter, on the other hand, heat shock is applied to the host cell into which the plasmid for transformation has been introduced at a desired timing during the culture of the host cell, so that the expression of the endonuclease can be induced at the desired timing.

Furthermore, in the aforementioned plasmid for transformation, when the pair of homologous recombination sequences have high homology to the upstream region and the downstream region of a predetermined gene, a fragment comprising a gene of interest is incorporated into the genome via homologous recombination, and, at the same time, the predetermined gene is deleted from the genome. By observing a phenotype resulting from the deletion of the predetermined gene, accordingly, whether or not the nucleic acid fragment comprising a gene of interest has been incorporated into the genome can be determined. When an ADE1 gene is utilized as such a predetermined gene, for example, the ADE1 gene is deleted from the genome if the nucleic acid fragment comprising a gene of interest is incorporated into the genome. As a result, 5-aminoimidazole riboside is accumulated in the host, and a transformant is colored red due to the polymerized polyribosylaminoimidazole. By detecting this red color, accordingly, it can be determined that the nucleic acid fragment comprising a gene of interest has been incorporated into the genome of the host.

It is to be noted that, in the aforementioned example, the plasmid for transformation is configured to comprise an inducible promoter and an endonuclease gene, but that the plasmid for transformation according to the present invention may also be configured not to have such an inducible promoter and an endonuclease gene. In this case, an expression vector comprising an inducible promoter and an endonuclease gene may be prepared separately, and the expression vector may be introduced into a host cell together with the plasmid for transformation according to the present invention. Even in this case, in the host cell into which the expression vector comprising an inducible promoter and an endonuclease gene and the plasmid for transformation having a gene of interest have been introduced, the endonuclease gene is expressed under the control of the inducible promoter, so that, as shown in FIG. 3, a nucleic acid fragment comprising a gene of interest sandwiched with a pair of homologous recombination sequences can be cleaved out, and a transformant in which the gene of interest is incorporated into the genome can be produced. With the use of a host cell into which an inducible promoter and an endonuclease gene have been introduced in advance, a plasmid for transformation may not comprise an inducible promoter and an endonuclease gene.

The transformation method and the method of producing a transformant using the plasmid for transformation according to the present invention are not particularly limited, and these methods can be applied to all types of host cells. Examples of the host cells include: fungi such as filamentous fungi and yeasts; bacteria such as Escherichia coli and Bacillus subtilis; plant cells; and animal cells including mammals and insects. The type of the yeast is not particularly limited, and examples thereof include yeasts belonging to the genus Saccharomyces, yeasts belonging to the genus Kluyveromyces, yeasts belonging to the genus Candida, yeasts belonging to the genus Pichia, yeasts belonging to the genus Schizosaccharomyces, and yeasts belonging to the genus Hansenula. More specifically, the aforementioned methods can be applied to yeasts belonging to the genus Saccharomyces such as Saccharomyces cerevisiae, Saccharomyces bayanus, or Saccharomyces boulardii. The type of the bacteria is not particularly limited, and examples thereof include bacteria belonging to the genus Bacillus, the genus Streptomyces, the genus Escherichia, the genus Thermus, the genus Rhizobium, the genus Lactococcus, and the genus Lactobacillus.

In the method of transformation and the method of producing a transformant using the plasmid for transformation according to the present invention, in particular, the plasmid for transformation comprises a counter selection marker. Upon expression of the counter selection marker, a host cell in which a gene of interest remains uncleaved and incorporated in a circular plasmid can be induced to die. As shown in FIG. 1 or 2, when the plasmid for transformation according to the present invention is used, there is a case that the gene of interest may not be incorporated into genome DNA. The gene of interest may be present in the form of a circular plasmid in the host cell. If the plasmid for transformation does not comprise a counter selection marker and a transformed cell may be selected based on expression of the gene of interest or a selection marker introduced together with the gene of interest, a cell in which the gene of interest is not incorporated into genome DNA but is present in the form of a circular plasmid is selected (false-positive cell).

When the gene of interest is cleaved by an endonuclease, as shown in FIG. 3, a plasmid for transformation becomes linearized. Thus, the plasmid would not be replicated and detached as the cell grows. When the gene of interest is cleaved by the endonuclease, accordingly, a positive cell can be selected based on expression of the gene of interest or a selection marker introduced together with the gene of interest without the influence of the counter selection marker.

### EXAMPLES

Hereinafter, the present invention will be described in greater detail with reference to the following examples. However, these examples are not intended to limit the technical scope of the present invention.

### Example 1

Method

1. Test Strain

An E. coli strain, NEB Turbo Competent E. coli (NEB), was used as a test line.

2. Production of Vector to be Introduced into E. coli Genome

The vector produced was an E. coli-yeast shuttle vector pUCtetR-P_tetA-SCEI-sacB-Ec araB-GFP-SmR-Ec araA-sacB comprising the I-SceI gene of the homing endonuclease derived from S. cerevisiae induced by tetracycline (SCEI), the sacB gene derived from Bacillus subtilis for counter selection (NCBI Accession Number: 936413), and a sequence formed by inserting a DNA fragment comprising homologous recombination sequences to be introduced into the genome between two recognition sequences of I-SceI (see FIG. 2). pUC-tetR-P_tetA-SCEI-sacB-Ec araBGFP-SmR-Ec araA-sacB comprises: the Tet repressor gene derived from transposon Tn10 (NCBI Accession Number: AP000342) (tetR); the SCEI gene linked to the tetA promoter inducible by tetracycline; an ampicillin resistance gene; the araB gene sequence and the araA gene sequence of the E. coli MG1655 strain (NCBI Accession Number: NC_000913.3) as homologous recombination sequences for genome introduction; as a gene to be introduced via homologous recombination, a GFP homologous gene (the gene does not comprise a sequence necessary for gene expression such as a promoter sequence and the gene is not expressed; NCBI Accession Number: MI085862); as a homologous recombination marker gene, a gene sequence comprising a spectinomycin resistance gene (the smR marker; NCBI Accession Number: No. X12870); and a the sacB gene, inserted into the pUC19 vector (see FIG. 2). This vector was composed of a region resulting from removal of the P_LtetO promoter, a yeast autonomous replication sequence (ARS), and a centromere sequence (CEN) from the separately produced pRScen-tetR-P_LtetO-SCEI-Ec araB-GFP-SmR-Ec araA vector (see Reference Example 1 below). It should be noted that araB, araA, a GFP homologous gene, and an smR marker sequence are inserted into a region between two homing endonuclease I-SceI cleavage recognition sequences, and such region can be cleaved upon expression of the SCEI gene added to the tetA promoter induced in a tetracycline-containing medium. A fragment cleaved in an E. coli cell is introduced into the genome via homologous recombination (see FIG. 3).

Individual DNA sequences can be amplified by PCR. To connect individual DNA fragments to one another, primers were synthesized to have a DNA sequence so as to overlap with a DNA sequence adjacent thereto by approximately 15 bp (Table 1). Using these primers, a DNA fragment of interest was amplified with the use of pRScen-tetR-P_LtetO-SCEI-Ec araB-GFP-SmR-Ec araA or a synthetic DNA sequence as a template, and the DNA fragments were connected to one another using the InFusion HD Cloning Kit or the like to produce a final vector of interest.

TABLE 1

| Amplified DNA fragment | Primer sequence (5′-3′) | SEQ ID NO: |
|---|---|---|
| Fragment comprising tetA promoter | TAATCTAGACATCATCATTAATTCCTAATTTTTGTTG | 1 |
| | ACACTCTATCATTGATAGAGTTATTTTACCAC | |
| | TTCTTAATGTTTTTCATTTCACTTTTCTCTATCACTG | 2 |
| | ATAGGGAGTGGTAAAATAACTCTATCAATGAT | |
| Fragment comprising SCEI gene, I-SceI | TGAAAAACATTAAGAAAAACCAAGTTATG | 3 |
| recognition sequence, araB gene, GFP | AGGACGGTGGCCGTTCTAAAG | 4 |
| homologous gene, smR gene, araA gene, | | |
| and I-SceI recognition sequence | | |
| Fragment comprising SacB gene | AACGGCCACCGTCCTCACATATACCTGCCGTTCAC | 5 |
| | AATAGGGGTTCCGCGTGTGCATGATCTCCTCGAAAAG | 6 |
| Fragment comprising ampicilin resistance | CGCGGAACCCCTATTTGTTTATTTTTC | 7 |
| gene, pUC replication origin, and tetR | ATGATGTCTAGATTAGATAAAAGTAAAGTGATTAACA | 8 |
| gene | G | |

3. Method of homologous recombination of E. coli genome and verification of introduction efficiency pUC-tetR-P_tetA-SCEI-sacB-Ec araB-GFP-SmR-Ec araA-sacB was used to transform NEB Turbo Competent E. coli, and plasmid-introduced colonies were selected with the aid of ampicillin. Subsequently, the plasmid-introduced strains were applied on an LB medium comprising spectinomycin and anhydrotetracycline (50 ng/ml) to induce homing endonuclease, a DNA fragment between homing endonuclease ISceI cleavage recognition sequences was cleaved, and the colonies homologously recombined with genome DNA were selected using a spectinomycin marker for homologous recombination. In addition, the selected colonies were subjected to counter selection in an LB medium comprising 10% sucrose, spectinomycin, and anhydrotetracycline (50 ng/ml). It is known that Levansucrase, which is a sacB gene product used for counter selection, converts sucrose into levan, levan is accumulated in a periplasm layer, and the cell is then induced to die. In the absence of sucrose, no lethality is observed. Thus, a vector comprising the sacB gene can be removed on the basis of the presence or absence of sucrose.

The grown colonies were subjected to PCRs amplifying a region between the E. coli genome and a DNA fragment incorporated via homologous recombination (the primers combination A shown in FIG. 3) and a region between both sides of the genome sandwiching the DNA fragment incorporated via homologous recombination (the primers combination B shown in FIG. 3). Colonies in which amplified bands were detected via both PCRs, in case that the primers combination B were used, the lengths of amplified fragment had increased by the length of the DNA fragments introduced and the bands having wild-type lengths had disappeared were counted as colonies resulting from homologous recombination of the genome. Colonies in which amplified bands were detected via both PCRs and in case that the primers combination B were used, the lengths amplified fragment had increased by the length of the DNA fragments introduced and the bands having wild-type lengths had been detected were counted as colonies contaminated with nonrecombinant cells. Colonies in which the bands having wild-type lengths had been selectively detected were counted as false-positive colonies. The efficiency for obtaining homologous recombinant colonies was then calculated. Table 2 shows the sequences of the primers used.

TABLE 2

| Primer | | SEQ |
|---|---|---|
| combination | Primer sequence (5′-3′) | ID NO: |
| A | CAAGCAGATTTATCGCCAGC | 9 |
| | TGGACGGCAGCTGATCCTGCCAGG | 10 |
| B | CAAGCAGATTTATCGCCAGC | 11 |
| | GTTGGGTGACCTGACGCAG | 12 |

### Results and Discussion

In this example, 40 colonies maintaining spectinomycin resistance after induction of the homing endonuclease by tetracycline were selected from among the strains into which the pUC-tetR-P_tetA-SCEI-sacB-Ec araB-GFP-SmR-Ec araA-sacB vector had been introduced. In addition, counter selection was performed using the sacB gene, and colonies were subjected to PCR before and after counter selection to inspect the homologous recombination efficiency (Table3). As a result of the test, the percentage of false-positive colonies was found to exceed 50% before counter selection. This indicates that a DNA fragment between the homing endonuclease I-SceI cleavage recognition sequences is not incorporated into genome DNA and there are many host cells in which circular plasmids for transformation remain (colonies contaminated with nonrecombinant cells). Colonies contaminated with nonrecombinant cells are considered to result from homologous recombination of colonies gradually occurred in the process of colony growth. Thus, the process of concentration of the cells resulting from homologous recombination of the genome via counter selection is considered to be an effective method for elimination of contamination with nonrecombinant cells.

TABLE 3

| | Colonies resulting | Colonies | | Colonies |
|---|---|---|---|---|
| | from homologous | contaminated with | | died |
| | recombination of | nonrecombinant | False-positive | after counter |
| | the genome | cells | colonies | selection |
| Before counter selection | 17.5% | 35% | 52.5% | — |
| After counter selection | 47.5% | 7.5% | 0% | 45.0% |

### Reference Example 1

In Reference Example 1, a method of producing the pRScen-tetR-P_LtetO-SCEI-Ec araB-GFP-SmR-Ec araA vector used in the above example is described. In order to produce the pRScen-tetR-P_LtetO-SCEI-Ec araB-GFP-SmR-Ec araA vector, at the outset, the pRS436(SAT)-P_GAL1-SCEI-T_CYC1-Sce-5U_ADE1-P_AgTEF1-G418-T_AgTEF1-3U_ADE1-Sce vector was produced. From the vector produced above, subsequently, the pRS436cen(SAT)-P_GAL1-SCEI-T_CYC1-Sce-5U_ADE1-P_AgTEF1-G418-T_AgT EF1-3U_ADE1-Sce vector was produced. The pRScen-tetR-P_LtetO-SCEI-Ec araBGFP-SmR-Ec araA vector was then produced from the pRS436cen(SAT)-P_GAL1-SCEI-T_CYC1-Sce-5U_ADE1-P_AgTEF1-G418-T_AgT EF1-3U_ADE1-Sce vector.

<Production of the pRS436(SAT)-P_GAL1-SCEI-T_CYC1-Sce-5U_ADE1-P_AgTEF1-G418-T_AgTEF1-3U_ADE1-Sce vector>

YEp-type yeast shuttle vectors, namely, pRS436(SAT)-P_MET25-SCEI-T_CYC1-Sce-5U_ADE1-P_AgTEF1-G418-T_AgTE F1-3U_ADE1-Sce and pRS436(SAT)-P_GAL1-SCEI-T_CYC1-Sce-5U_ADE1-P_AgTEF1-G418-T_AgTEF 1-3U_ADE1-Sce, each of which is constituted with a sequence formed by inserting S. cerevisiae-derived homing endonuclease I-SceI induced under methionine-deficient conditions or by galactose (SCEI gene; NCBI Accession Number: 854590) and a DNA fragment containing a pair of homologous recombination sequences to be introduced into the genome between a pair of I-SceI target sequences (endonuclease target sequences) were produced.

Regarding pRS436(SAT)-P_MET25-SCEI-T_CYC1-Sce-5U_ADE1-P_AgTEF1-G418-T_AgTE F1-3U_ADE1-Sce, a SCEI gene to which a MET25 promoter and a CYC1 terminator had been added (a sequence into which the intron of a COX5B gene had been inserted, and in which codons in the whole length had been converted depending on the codon use frequency in the nuclear genome of the yeast), a gene sequence containing a nourseothricin resistance gene (nat marker), as homologous recombination sequences to be introduced into the genome, the gene sequence in a region approximately 1000 bp upstream of the 5′-terminal side of an ADE1 gene (5U_ADE1) and the DNA sequence in a region approximately 950 bp downstream of the 3′-terminal side of the ADE1 gene (3U_ADE1), and as a marker gene for homologous recombination, a gene sequence containing a G418 resistance gene (G418 marker), to which Ashbya gossypii-derived TEF1 promoter and TEF1 terminator had been added were inserted into a vector prepared by removing a URA3 gene, a TDH3 promoter, and a CYC1 terminator from the pRS436GAP vector (NCBI Accession Number: AB304862).

Individual DNA sequences can be amplified by PCR. To connect individual DNA fragments to one another, primers were synthesized to have a DNA sequence so as to overlap with a DNA sequence adjacent thereto by approximately 15 bp (Table 4). Using these primers, a DNA fragment of interest was amplified with the use of the genome of the S. cerevisiae OC-2 strain or synthetic DNA as a template, and the DNA fragments were connected to one another using the In-Fusion HD Cloning Kit or the like. The resultant was cloned into the pRS436GAP vector to produce a final plasmid of interest.

In the pRS436(SAT)-P_GAL1-SCEI-T_CYC1-Sce-5U_ADE1-P_AgTEF1-G418-T_AgTEF 1-3U_ADE1-Sce of interest, a SCEI gene to which a GAL1 promoter had been added instead of a MET25 promoter was inserted. The SCEI gene can be expressed in a medium containing galactose as a carbon source, and a sequence inserted between ISceI target sequences can be cleaved. This vector was produced by amplifying DNA fragments of interest using pRS436(SAT)-P_MET25-SCEI-T_CYC1-Sce-5U_ADE1-P_AgTEF1-G418-T_AgTE F1-3U_ADE1-See or the genome of the S. cerevisiaeOC-2 strain as a template (the used primers are shown in Table 4) and then connecting the DNA fragments to one another using the In-Fusion HD Cloning Kit or the like.

TABLE 4

| | | |
|---|---|---|
| | | SEQ ID |
| Amplified DNA fragment | Primer sequence (5′-3′) | NO: |

pRS436(SAT)-P_MET25-SCEI-T_CYC1-Sce-5U_ADE1-P_AgTEF1-G418-T_AgTEF1-3U_ADE1-Sce

| | | |
|---|---|---|
| MET25 promoter | ATAATATACTAGTAACGTAAATACTAGTTAGTAGATGATAGTTG | 13 |
| | TGTATGGATGGGGGTAATAGAATTG | 14 |
| COX58 intron | ACCCCCATCCATACAAGCATGTATAACAAACACTGATTTTTG | 15 |
| | TCTTAATGTTTTTCACTGCAAAACTTGTGCTTGTACAC | 16 |
| SCEI | TGAAAAACATTAAGAAAAACCAAGTTATG | 17 |
| | GCGTGACATAACTAATCATTTCAAGAAGGTTTCGGAG | 18 |
| CYC1 terminator (including | TTAGTTATGTCACGCTTACATTCACG | 19 |
| I-SceI target sequence) | AATTGCCCGACTCATATTACCCTGTTATCCCTAAGCTTGCAAATTAAAGC | 20 |
| | CTTCGAGCG | |
| 5U_ADE1 | ATGAGTCGGGCAATTCCG | 21 |
| | CTGGGCCTCCATGTCTATCGTTAATATTTCGTATGTGTATTCTTTG | 22 |
| TEF1 promoter derived | GACATGGAGGCCCAGAATAC | 23 |
| from Ashbya gossypii | GGTTGTTTATGTTCGGATGTGATG | 24 |
| G418 | CGAACATAAACAACCATGGGTAAGGAAAAGACTCACGTTTC | 25 |
| | TATTGTCAGTACTGATTAGAAAAACTCATCGAGCATCAAATGAAAC | 26 |
| TEF1 terminator derived | TCAGTACTGACAATAAAAAGATTCTTGTTTTCAAG | 27 |
| from Ashbya gossypii | CAGTATAGCGACCAGCATTCACATACG | 28 |
| TEF1 promoter (including | ATAGCATACATTATACGAAGTTATCCCACACACCATAGCTTCAAAATG | 29 |
| part of LoxP sequence) | CACCGAAATCTTCATCCCTTAGATTAGATTGCTATGC | 30 |
| 3U_ADE1 (including I-SceI | GCTGGTCGCTATACTGCGTGATTTACATATACTACAAGTCG | 31 |
| target sequence) | AAAAACATAAGACAAATTACCCTGTTATCCCTATGACCGGATGAAACC | 32 |
| pRS436 (including 2 μ | GGGATAACAGGGTAATGGTACCCAATTCGCCCTATAG | 33 |
| replication origin) | TACCGCACAGATGCGTAAGG | 34 |
| LEU2 terminator | TTACGCATCTGTGCGGTAAGGAATCATAGTTTCATGATTTTCTG | 35 |
| | CAGGATGACGCCTAAAAAGATTCTCTTTTTTTATGATATTTGTAC | 36 |
| nourseothricin resistance | TTAGGCGTCATCCTGTGCTC | 37 |
| gene | CACACTAAATTAATAATGAAGATTTCGGTGATCCC | 38 |
| CYC1 promoter | TATTAATTTAGTGTGTGTATTTGTGTTTGTGTG | 39 |
| | GCAGATTGTACTGAGAGTACGACATCGTCGAATATGATTC | 40 |
| pRS436 (including | ACTCTCAGTACAATCTGCTCTGATGC | 41 |
| ampicillin resistance gene | TTACTAGTATATTATGCTCCAGCTTTTGTTCCCTTTAG | 42 |
| and ColE1 replication | | |
| origin) | | |

pRS436(SAT)-P_GAL1-SCEI-T_CYC1-Sce-5U_ADE1-P_AgTEF1-G418-T_AgTEF1-3U_ADE1-Sce

| | | |
|---|---|---|
| Sequence other than GAL1 | TCAAGGAGAAAAAACCAGCATGTATAACAAACACTGATTTTTGTTTTG | 43 |
| promoter | CGGCTTCTAATCCGTGCTCCAGCTTTTGTTCCCTTTAG | 44 |
| GAL1 promoter | ACGGATTAGAAGCCGCCGAG | 45 |
| | GGTTTTTTCTCCTTGACGTTAAAGTATAG | 46 |

<Production of the pRScen-tetR-P_LtetO-SCEI-Ec araB-GFP-SmR-Ec araA vector>The pRS436cen(SAT)-P_GAL1-SCEI-T_CYC1-Sce-5U_ADE1-P_AgTEF1-G418-T_AgT EF1-3U_ADE1-Sce vector used for producing the pRScen-tetR-P_LtetO-SCEI-Ec araB-GFP-SmR-Ec araA vector is a vector in which a 2-microM plasmid-derived replication origin is deleted from the pRS436(SAT)-P_GAL1-SCEI-T_CYC1-Sce-5U_ADE1-P_AgTEF1-G418-T_AgTEF1-3U_ADE1-Sce vector described above and, instead thereof, an autonomous replication sequence (ARS) and a centromere sequence (CEN) are inserted therein. The copy number of the vector in a cell is retained to be one.

The pRScen-tetR-P_LtetO-SCEI-Ec araB-GFP-SmR-Ec araA vector is an E. coliyeast shuttle vector composed of a sequence comprising the I-SceI gene of the homing endonuclease derived from S. cerevisiae induced by tetracycline (SCEI) and a DNA fragment comprising homologous recombination sequences to be introduced into the genome inserted between two recognition sequences of I-SceI. pRScentetR-P_LtetO-SCEI-Ec araB-GFP-SmR-Ec araA comprises: the Tet repressor gene derived from transposon Tn10 (NCBI Accession Number: AP000342) (tetR); the SCEI gene linked to the tetracycline-inducible LtetO-1 promoter (Lutz, R. and Bujard, H., “Independent and Tight Regulation of Transcriptional Units in Escherichia Coli Via the LacR/O, the TetR/O and AraC/I1-I2 Regulatory Elements,” Nucleic Acids Research, 25, 1997: 1203-1210); an ampicillin resistance gene; homologous recombination sequences to be introduced into the genome (i.e., the araB gene sequence and the araA gene sequence of the E. coli MG1655 strain (NCBI Accession Number: NC_000913.3)); a GFP homologous gene to be introduced via homologous recombination (the gene does not comprise a sequence necessary for gene expression such as a promoter sequence and the gene is not expressed; NCBI Accession Number: MI085862); and a gene sequence comprising a spectinomycin resistance gene (the smR marker; NCBI Accession Number: No. X12870) as a homologous recombination marker gene, inserted in the yeast shuttle vector. This yeast shuttle vector was composed of a region resulting from removal of GAL1 promoter, CYC1 terminator, ADE1 5′ homologous recombination sequence, a G418 marker gene, and the ADE1 3′ homologous recombination sequence from the separately produced pRS436cen(SAT)-P_GAL1-SCEI-T_CYC1-Sce-5U_ADE1-P_AgTEF1-G418-T_AgT EF1-3U_ADE1-Sce vector.

Individual DNA sequences can be amplified by PCR. To connect individual DNA fragments to one another, primers were synthesized to have a DNA sequence so as to overlap with a DNA sequence adjacent thereto by approximately 15 bp (Table 5). Using these primers, a DNA fragment of interest was amplified with the use of the genome of the MG1655 strain, pRS436cen(SAT)-P_GAL1-SCEI-T_CYC1-Sce-5U_ADE1-P_AgTEF1-G418-T_AgT EF1-3U_ADE1-Sce, the TEF-Dasher GFP plasmid (ATUM), or a synthetic DNA sequence as a template, and the DNA fragments were connecte to one another using the In-Fusion HD Cloning Kit or the like to produce a final vector of interest.

TABLE 5

| | | SEQ ID |
|---|---|---|
| Amplified DNA fragment | Primer sequence (5′-3′) | NO: |
| Fragment comprising tetR | CATGTTCTTTCCTGCGTTATTAAGACCCACTTTCACATTTAAGTTGTTTT | 47 |
| gene | TC | |
| | CTATCACTGATAGGGAGATTTTCACTTTTCTCTATCACTGATAGG | 48 |
| Fragment comprising | TCCCTATCAGTGATAGAGATTGACATCCCTATCAGTGATAGAGATACTGA | 49 |
| LtetO-1 promoter | GCACATCAGCAGGACGCAC | |
| | AAGTTAAACAAAATTATTTCTAGCTTTCTCCTCTTTAATGAATTCGGTCA | 50 |
| | GTGCGTCCTGCTGATGTGC | |
| Fragment comprising | GAAATAATTTTGTTTAACTTTAAGAAGGAGATATACATAATGAAAAACAT | 51 |
| SECI gene | TAAGAAAAACCAAGTTATG | |
| | TAGGCCAGTACCTCCCGCTTATGTATTTACTCGTAGGATTTGCTTCGTTC | 52 |
| | GATCAGCACAAGCCTTCAAAGATGATCATTTCAAGAAGGTTTCGGAGGAG | |
| Fragment comprising araB | GGGAGGTACTGGCCTAGCGTCGTGGCCCGGGAGAGACAGTTTAGTAGTGA | 53 |
| gene and I-SceI | CTCGCGGCCAGTTAGGGATAACAGGGTAATATGGCGATTGCAATTGGC | |
| recognition sequence | CAATCACAGGGCGGGAAATAAGCTACAATTAACGCCAAAAAATTATAGAG | 54 |
| | TCGCAACGGCC | |
| Fragment comprising GFP | TCCCGCCCTGTGATTGAGGGGGGATGGTGTCCCCACAGTATGACCGCACT | 55 |
| homologous gene | AACAGAAGG | |
| | CATACATTTCTCCACGGGACCCACAGTCGTAGATGCGTAAAATCAACCTT | 56 |
| | GGTAAGTATCCAAATCC | |
| Fragment comprising smR | CGTGGAGAAATGTATGAAACCCTGTATGGAGAGTGATTCAGTCCAGCCAG | 57 |
| gene | GACAGAAATG | |
| | TTCTCCCAAGTGTACGATATCACACCTAGCGCCGTGCAAAAAAAACCACG | 58 |
| | TCAAATAATCAAGGCGCCTTGAATGCTCGAGGGTTATTTGCCGACTACCT | |
| | TGGTG | |
| Fragment comprising araA | CGTACACTTGGGAGAAGTCAGATACGATTGCGGCTCAGTATGACGATTTT | 59 |
| gene and I-SceI | TGATAATTATGAAGTGTGGTTTG | |
| recognition sequence | CGGCAGTACCGGATCCTAAAGCCGATTCAAGAAAAATTACCCTGTTATCC | 60 |
| | CTATTAGCGACGAAACCCGTAATAC | |
| Fragment comprising pUC | GGATCCGGTACTGCCGACGCACTTTAGAACGGCCACCGTCCTGGTCCTTT | 61 |
| replication origin, | TCATCACGTGC | |
| ampicilin resistance gene, | TAACGCAGGAAAGAACATGTGAGC | 62 |
| autonomously replicated | | |
| sequence (ARS), and | | |
| centromere sequence | | |
| (CEN) | | |

### Example 2

Method

1. Test Strain

A monoploid experimental yeast, S. cerevisiae BY4742, was used as a test yeast line.

2. Production of Vector to be Introduced into Yeast Genome

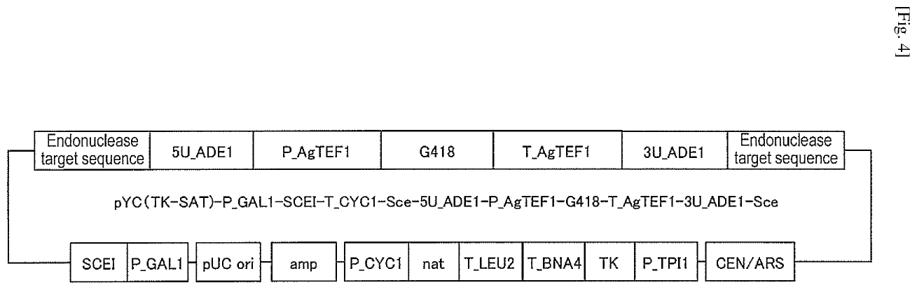

The vector produced was the YCp-type yeast shuttle vector pYC(TK-SAT)-P_GAL1-SCEI-T_CYC1-Sce-5U_ADE1-P_AgTEF1-G418-T_AgTEF 1-3U_ADE1-Sce comprising S. cerevisiae-derived homing endonuclease I-SceI induced by galactose (SCEI gene), a thymidine kinase as a counter selection marker, and a sequence formed by inserting a DNA fragment comprising homologous recombination sequences to be introduced into the genome between two recognition sequences of I-SceI (FIG. 4). pYC (TK-SAT)-P_GAL1-SCEI-T_CYC1-Sce-5U_ADE1-P_AgTEF1-G418-T_AgTEF1-3U_ADE1-Sce comprises a SCEI gene to which a GAL1 promoter and a CYC1 terminator had been added (a sequence into which the intron of a COX5B gene had been inserted, and in which codons in the whole length had been converted depending on the codon use frequency in the nuclear genome of the yeast), the herpes simplex virus type 1 thymidine kinase gene to which a TPI1 promoter and a BNA4 terminator had been added (a sequence in which codons in the whole length had been converted depending on the codon use frequency in the nuclear genome of the yeast; “TK” in FIG. 4), as homologous recombination sequences to be introduced into the genome, the gene sequence in a region approximately 1000 bp upstream of the 5′-terminal side of an ADE1 gene (5U_ADE1) and the DNA sequence in a region approximately 950 bp downstream of the 3′-terminal side of the ADE1 gene (3U_ADE1), and as a marker gene for homologous recombination, a gene sequence comprising a G418 resistance gene (G418 marker) to which Ashbya gossypii-derived TEF1 promoter and TEF1 terminator had been added. 5U_ADE1, 3U_ADE1, and the G418 marker sequence are inserted into a region between two homing endonuclease I-SceI recognition sequences, and a region comprising the same can be cleaved with the aid of the SCEI gene added to the GAL1 promoter that is induced in a medium containing galactose as a carbon source.

Individual DNA sequences can be amplified by PCR. To connect individual DNA fragments to one another, primers were synthesized to have a DNA sequence so as to overlap with a DNA sequence adjacent thereto by approximately 15 bp (Table 6). Using these primers, a DNA fragment of interest was amplified with the use of pRS436cen(SAT)-P_GAL1-SCEI-T_CYC1-Sce-5U_ADE1-P_AgTEF1-G418-T_AgT EF1-3U_ADE1-Sce (Reference Example 2 below), the genome of the S. cerevisiae OC-2 strain, or synthetic DNA as a template, and the DNA fragments were connected to one another using the In-Fusion HD Cloning Kit or the like to produce a plasmid of interest.

TABLE 6

| | | SEQ ID |
|---|---|---|
| Amplified DNA fragment | Primer sequence (5′-3′) | NO: |
| TPI1 promoter | GGCAAGCGATCCGTCCTAGGCAAGAGAGAAGACCCAGAGATGTTG | 63 |
| | AGGATAACTGGCCATTTTTAGTTTATGTATGTGTTTTTTGTAGTTATAGA | 64 |
| | TTTAAG | |
| TK gene | ATGGCCAGTTATCCTTGTCACC | 65 |
| | TCAATTAGCTTCCCCCATTTCTC | 66 |
| BNA4 terminator | GGGGAAGCTAATTGAGAGCCAGTTTATTCTTGCCATCC | 67 |
| | TGAAACTATGATTCCTCGATCAATGCGAAATTCCAACTATTTC | 68 |
| Sequences other than TPI1 | AGGAATCATAGTTTCATGATTTTCTGTTAC | 69 |
| promoter, TK gene, and | GGACGGATCGCTTGCCTGTAAC | 70 |
| BNA4 terminator | | |

3. Method of Homologous Recombination of Yeast Genome and Verification of Introduction Efficiency

Using the produced YC (TK-SAT)-P_GAL1-SCEI-T_CYC1-Sce-5U_ADE1-P_AgTEF1-G418-T_AgTEF1-3U_ADE1-Sce vector, the S. cerevisiae BY4742 strain was transformed, and the colonies, in which the plasmid was introduced, were selected in a G418-containing YPD agar medium according to the method of Akada et al. (Akada, R. et al., “Elevated temperature greatly improves transformation of fresh and frozen competent cells in yeast,” BioTechniques 28, 2000: 854-856). All the grown colonies were white colonies. Subsequently, the plasmid-introduced strains were applied to a G418-containing YPGa agar medium (carbon source: galactose), homing endonuclease was induced to express, a DNA fragment between the homing endonuclease I-SceI cleavage recognition sequences was cleaved, the colonies homologously recombined with genome DNA were selected with the aid of G418, and the ADE1 gene-disrupted strains were counted depending on coloration of colonies. The ADE1 gene is a gene of adenine biosynthesis pathway. In the ADE1 gene-disrupted strain, 5-aminoimidazole riboside as an intermediate metabolite of adenine is accumulated, and the polyribosylaminoimidazole polymerized with 5-aminoimidazole is colored red. Thus, the ADE1 gene-disrupted strain can be easily distinguished via visual observation. In addition, the white colony, the ADE1-nondisrupted strain, was applied to an SD medium containing 5-fluoro-2-deoxyuridine (50 microgram/ml) to inspect the growth. Since the thymidine kinase gene converts 5FU into a toxic metabolite, a cell having the thymidine kinase gene would be induced to die in a 5FU-containing medium. In the absence of 5FU, in contrast, the cell having the thymidine kinase gene would not be induced to die. By allowing such cell to grow in a medium containing 5FU, a plasmid containing a TK gene can be removed.

Results and Discussion

From the strain into which the pYC (TK-SAT)-P_GAL1-SCEI-T_CYC1-Sce-5U_ADE1-P_AgTEF1-G418-T_AgTEF1-3U_ADE1-Sce vector had been introduced as a plasmid, homing endonuclease was induced to express, and the efficiency of homologous recombination into the ADE1 gene locus was examined. The results demonstrate that efficiency of homologous recombination into the ADE1 gene locus was high, and false-positive white colonies were also developed, although the frequency of false-positive results was low (Table 7). The 3 false-positive colonies were transferred to a 5FU-containing medium and selected. As a result, 2 colonies died. It is highly likely that these two colonies could not be eliminated via counter selection due to retaining the plasmid without cleaving from the plasmid via homologous recombination. The remaining one colony underwent nonhomologous recombination with a region other than ADE1. On the basis of the results above, a step of concentration of cells resulting from homologous recombination of the genome via counter selection is considered to be an effective method of eliminating contamination with cells without genome recombination.

TABLE 7

| Efficiency for obtaining | Efficiency for obtaining |
|---|---|
| ADE1-disrupted strains | ADE1-undisrupted strains |
| (Red colonies) | (White colonies) |
| 9.6 × 10⁻³ | 3.0 × 10⁻⁴ |

### Reference Example 2

In Reference Example 2, a method of producing pRS436cen(SAT)-P_GAL1-SCEI-T_CYC1-Sce-5U_ADE1-P_AgTEF1-G418-T_AgT EF1-3U_ADE1-Sce used in the examples is described. pRS436cen(SAT)-P_GAL1-SCEI-T_CYC1-Sce-5U_ADE1-P_AgTEF1-G418-T_AgT EF1-3U_ADE1-Sce is a vector in which a 2-microM plasmid-derived replication origin is deleted from pRS436(SAT)-P_GAL1-SCEI-T_CYC1-Sce-5U_ADE1-P_AgTEF1-G418-T_AgTEF1-3U_ADE1-Sce and, instead thereof, an autonomous replication sequence (ARS) and a centromere sequence (CEN) are inserted therein. The copy number of the vector in a cell is retained to be one. This vector was produced by amplifying DNA fragments of interest using RS436(SAT)-P_GAL1-SCEI-T_CYC1-Sce-5U_ADE1-P_AgTEF1-G418-T_AgTEF1-3U_ADE1-Sce or the genome of the S. cerevisiae OC-2 strain as a template (the primers used are shown in Table 8) and connecting the DNA fragments to one another using the In-Fusion Kit or the like.

pRS436(SAT)-P_GAL1-SCEI-T_CYC1-Sce-5U_ADE1-P_AgTEF1-G418-T_AgTE F1-3U_ADE1-Sce comprises: a SCEI gene to which a GAL1 promoter and a CYC1 terminator had been added (a sequence into which the intron of a COX5B gene had been inserted, and in which codons in the whole length had been converted depending on the codon use frequency in the nuclear genome of the yeast); a gene sequence containing a nourseothricin resistance gene (nat marker); as homologous recombination sequences to be introduced into the genome, the gene sequence in a region approximately 1000 bp upstream of the 5′-terminal side of an ADE1 gene (5U_ADE1) and the DNA sequence in a region approximately 950 bp downstream of the 3′-terminal side of the ADE1 gene (3U_ADE1); and as a marker gene for homologous recombination, a gene sequence comprising a G418 resistance gene (G418 marker) to which Ashbya gossypii-derived TEF1 promoter and TEF1 terminator had been added, inserted into a vector prepared by removing a URA3 gene, a TDH3 promoter, and a CYC1 terminator from the YEp-type yeast shuttle vector pRS436GAP (NCBI Accession Number: AB304862). 5U_ADE1, 3U_ADE1, and the G418 marker sequence are inserted into a region between 2 homing endonuclease I-SceI recognition sequences, and such region can be cleaved by the SECI gene added to the GAL1 promoter inducible in a medium containing galactose as a carbon source.

Individual DNA sequences can be amplified by PCR. To connect individual DNA fragments to one another, primers were synthesized to have a DNA sequence so as to overlap with a DNA sequence adjacent thereto by approximately 15 bp (Table 8). Using these primers, a DNA fragment of interest was amplified with the use of the genome of the S. cerevisiae OC-2 strain or synthetic DNA as a template, and the DNA fragments were connected to one another using the In-Fusion HD Cloning Kit or the like. The resultant was cloned into the pRS436GAP vector to produce a final plasmid of interest.

TABLE 8

| | | |
|---|---|---|
| | | SEQ ID |
| Amplified DNA fragment | Primer sequence (5′-3′) | NO: |

pRS436(SAT)-P_GAL1-SCEI-T_CYC1-Sce-5U_ADE1-P_AgTEF1-G418-T_AgTEF1-3U_ADE1-Sce

| | | |
|---|---|---|
| GAL1 promoter | ACGGATTAGAAGCCGCCGAG | 71 |
| | GGTTTTTTCTCCTTGACGTTAAAGTATAG | 72 |
| COX5B intron | TCAAGGAGAAAAAACCAGCATGTATAACAAACACTGATTTTTGTTTTG | 73 |
| | TCTTAATGTTTTTCACTGCAAAACTTGTGCTTGTACAC | 74 |
| SCEI | TGAAAAACATTAAGAAAAACCAAGTTATG | 75 |
| | GCGTGACATAACTAATCATTTCAAGAAGGTTTCGGAG | 76 |
| CYC1 terminator (including | TTAGTTATGTCACGCTTACATTCACG | 77 |
| I-SceI target sequence) | AATTGCCCGACTCATATTACCCTGTTATCCCTAAGCTTGCAAATTAAAGC | 78 |
| | CTTCGAGCG | |
| 5U_ADE1 | ATGAGTCGGGCAATTCCG | 79 |
| | CTGGGCCTCCATGTCTATCGTTAATATTTCGTATGTGTATTCTTTG | 80 |
| TEF1 promoter derived from | GACATGGAGGCCCAGAATAC | 81 |
| Ashbya gossypii | GGTTGTTTATGTTCGGATGTGATG | 82 |
| G418 | CGAACATAAACAACCATGGGTAAGGAAAAGACTCACGTTTC | 83 |
| | TATTGTCAGTACTGATTAGAAAAACTCATCGAGCATCAAATGAAAC | 84 |
| TEF1 terminator derived | TCAGTACTGACAATAAAAAGATTCTTGTTTTCAAG | 85 |
| from Ashbya gossypii | CAGTATAGCGACCAGCATTCACATACG | 86 |
| TEF1 promoter (including | ATAGCATACATTATACGAAGTTATCCCACACACCATAGCTTCAAAATG | 87 |
| part of LoxP sequence) | CACCGAAATCTTCATCCCTTAGATTAGATTGCTATGC | 88 |
| 3U_ADE1 (including I-SceI | GCTGGTCGCTATACTGCGTGATTTACATATACTACAAGTCG | 89 |
| target sequence) | AAAAACATAAGACAAATTACCCTGTTATCCCTATGACCGGATGAAACC | 90 |
| pRS436 (including 2 μ | GGGATAACAGGGTAATGGTACCCAATTCGCCCTATAG | 91 |
| replication origin) | TACCGCACAGATGCGTAAGG | 92 |
| LEU2 terminator | TTACGCATCTGTGCGGTAAGGAATCATAGTTTCATGATTTTCTG | 93 |
| | CAGGATGACGCCTAAAAAGATTCTCTTTTTTTATGATATTTGTAC | 94 |
| nourseothricin resistance | TTAGGCGTCATCCTGTGCTC | 95 |
| gene | CACACTAAATTAATAATGAAGATTTCGGTGATCCC | 96 |
| CYC1 promoter | TATTAATTTAGTGTGTGTATTTGTGTTTGTGTG | 97 |
| | GCAGATTGTACTGAGAGTACGACATCGTCGAATATGATTC | 98 |
| pRS436 (including ampicillin | ACTCTCAGTACAATCTGCTCTGATGC | 99 |
| resistance gene and ColE1 | CGGCTTCTAATCCGTGCTCCAGCTTTTGTTCCCTTTAG | 100 |
| replication origin) | | |

pRS436cen(SAT)-P_GAL1-SCEI-T_CYC1-Sce-5U_ADE1-P_AgTEF1-G418-T_AgTEF1-3U_ADE1-Sce

| | | |
|---|---|---|
| Sequences other than GAL1 | TCAAGGAGAAAAAACCAGCATGTATAACAAACACTGATTTTTGTTTTG | 101 |
| promoter | CGGCTTCTAATCCGTGCTCCAGCTTTTGTTCCCTTTAG | 102 |
| GAL1 promoter | GGTCCTTTTCATCACGTGCTA | 103 |
| | GGTTTTTTCTCCTTGACGTTAAAGTATAG | 104 |

1. A plasmid for transformation comprising a site into which a gene of interest is to be incorporated, a pair of homologous recombination sequences sandwiching the site, a pair of endonuclease target sequences sandwiching the pair of homologous recombination sequences, and a counter selection marker.

2. The plasmid for transformation according to claim 1, which further comprises a target-specific endonuclease gene specifically cleaving the double strands of the endonuclease target sequences in an expressible manner.

3. The plasmid for transformation according to claim 2, wherein the target-specific endonuclease gene is a homing endonuclease gene.

4. The plasmid for transformation according to claim 3, wherein the endonuclease target sequence is specifically recognized by homing endonuclease.

5. The plasmid for transformation according to claim 2, which further comprises an inducible promoter regulating the expression of the target-specific endonuclease gene.

6. The plasmid for transformation according to any one of claim 1, which comprises the gene of interest that is incorporated into the site.

7. A method of producing a transformant, comprising steps of: introducing the plasmid for transformation according to claim 6 into a host; and selecting a transformant, in which the gene of interest comprised in the plasmid for transformation is incorporated into the genome of the host via the homologous recombination sequences comprised in the plasmid for transformation, and in which the gene of interest is then expressed therein,
wherein the counter selection marker functions to induce the death of a host comprising the plasmid for transformation comprising the gene of interest incorporated therein.

8. A transformation method comprising a step of introducing the plasmid for transformation according to claim 6 into a host, wherein the gene of interest comprised in the plasmid for transformation is expressed in the host and the counter selection marker functions to induce the death of a host comprising the plasmid for transformation comprising the gene of interest incorporated therein.

9. The transformation method according to claim 8, wherein the gene of interest is incorporated into the genome of the host via the homologous recombination sequences comprised in the plasmid for transformation.
